(19) United States
(12) Patent Application Publication
(10) Pub. No.: US 2008/0131463 A1
(43) Pub. Date: Jun. 5, 2008

(54) Vaccination method for controlling coccidiosis in fowl

(75) Inventors: Bruce Stewart-Brown, Salisbury, MD (US); Henry M. Engster, Salisbury, MD (US); Douglas K. Marvil, Laurel, DE (US)

Correspondence Address: VENABLE LLP, P.O. BOX 34385, WASHINGTON, DC, 20043-9998, US

(73) Assignee: Perdue Holdings, Inc., Wilmington, DE (US)

(21) Appl. No.: 11/606,571

(22) Filed: Nov. 30, 2006

(51) Int. Cl. A61K 39/012

(52) U.S. Cl. 424/271.1

(57) ABSTRACT

A method for protecting fowl against coccidiosis includes the steps of inoculating the fowl with coccidial oocysts from a strain of Eimeria that is at least partially resistant to an ionophore and subsequently administering to the fowl the ionophore and at least one chemotherapeutic anticoccidial agent. In particular, the ionophore is administered in stages both before and after the administration of the at least on chemotherapeutic agent. In a preferred embodiments, the method provides protection from coccidiosis without administration of antibiotics to the fowl and without continued administration of agents throughout the life of the fowl.

### FIELD OF THE INVENTION

The invention relates to immunizing fowl against diseases caused by coccidia, and involves the administration of an Eimeria strain and various agents to the fowl.

### BACKGROUND OF THE INVENTION

Coccidiosis is an economically important parasitic disease of fowl that depresses weight gain and feed efficiency in fowl. Coccidiosis is caused by infection of fowl with intracellular protozoal parasites of the genus Eimeria. Species of Eimeria that cause coccidiosis in chickens include E. acervulina, E. maxima, E. mitis, E. mivati, E. necatrix, and E. tenella. The infective stage of the Eimeria parasite is the sporulated oocyst, which ruptures upon ingestion, releasing four sporocysts into the intestinal tract. Each sporocyst releases two mobile sporozoites that penetrate the intestinal epithelial cells and reproduce. Newly formed oocysts are excreted in fecal droppings and infect new hosts, spreading infection rapidly throughout a population of fowl.

Coccidiosis in fowl has been treated with coccidiostatic agents, such as ionophores; however, these agents have become less effective as the coccidial parasites become resistant to them. As a result, treatment programs have been developed to vaccinate fowl against the parasites. Immunization creates a controlled coccidial infection throughout the life of the fowl to prevent the spread of coccidiosis and control the severity of the infection. For example, as described in U.S. Pat. No. 4,935,007, which is incorporated herein by reference, a strain of coccidial parasite that is sensitive to a particular agent or drug is administered to vaccinate the fowl against subsequent infection with drug-resistant strains of coccidial parasites, and drug treatment is used throughout the life of the fowl to control the virulence of the infection. An alternative method is to vaccinate fowl with less pathogenic, attenuated strains of Eimeria as described in U.S. Pat. No. 6,908,620, incorporated herein by reference.

### SUMMARY OF THE INVENTION

A method for protecting a fowl against coccidiosis comprising the steps of (a) orally inoculating a neonatal fowl with a suspension of sporulated coccidial oocysts, wherein the coccidial oocysts are from a strain of Eimeria that is at least partially resistant to an ionophore; (b) subsequently administering to the inoculated fowl the ionophore to which the Eimeria strain is at least partially resistant; (c) continuing to administer the ionophore to the inoculated fowl daily for up to 21 days; (d) subsequently administering at least one chemotherapeutic anticoccidial agent to the inoculated fowl for 3 to 10 days; and (e) subsequently administering the ionophore to the inoculated fowl daily for at least 7 days.

It is to be understood that both the foregoing general description and the following detailed description are exemplary, but are not restrictive, of the invention.

### DETAILED DESCRIPTION OF THE INVENTION

The present invention provides an immunological method to control coccidiosis in fowl. The method comprises a vaccination program utilizing a strain of Eimeria that shows at least partial resistance to a particular ionophore, and preferably short-term ionophore administration instead of ionophore treatment throughout the life of the fowl to maintain protection against a coccidial infection. For purposes of this invention, “fowl” includes chickens, turkeys, ducks, geese, quail, and pheasants. The vaccination program is especially well-suited to controlling coccidiosis in poultry, most particularly chickens.

The method comprises orally inoculating fowl at the neonatal (hatchling) stage, e.g., within about 48 hours of hatching, and preferably within 12-24 h of hatching, by orally administering to the fowl a suspension of sporulated oocysts from a strain of Eimeria that is at least partially resistant to a particular ionophore. Eimeria species used for vaccination include, but are not limited to, E. acervulina, E. maxima, E. mitis, E. mivati, E. necatrix, and E. tenella. Methods of preparation of infective sporulated oocysts are known to those of ordinary skill in the art (for example, see U.S. Pat. No. 4,935,007).

Oral administration is preferred, as administration by other routes is less certain to engender the necessary immunological response. Oral administration is also desirable in that the number of infective organisms administered can be controlled. Any effective method of oral administration may be used, such as directly spraying or placing a suspension of sporulated oocysts into the mouth of the bird, or administering the sporulated oocysts by aerosol delivery across the posterior surface of the bird. Aerosol delivery is an indirect mode of delivery to the mouth cavity. Material deposited from an aerosol onto a bird will be ingested by the bird (or another bird) through preening, and any material absorbed from the aerosol ocularly or nasally will drain to the mouth cavity.

The exact number of infective coccidial organisms to be administered is not critical, but the number must be effective to engender an immunological response by the fowl. The vaccine suspension preferably contains from 10-10,000 sporulated coccidial oocysts per hatchling, more preferably 100-1,000 oocysts. The concentration of oocysts in the suspension depends on the species of Eimeria utilized, for example, 400 oocysts per hatchling is a preferred dose for E. maxima, and 300 oocysts per hatchling is a preferred dose for E. acervulina. Effective doses of Eimeria species are known, or can be easily determined, by those of ordinary skill in the art.

Following administration of a suspension of Eimeria coccidial oocysts, an ionophore, to which the Eimeria species selected as the inoculant is at least partially resistant, is administered to the fowl, preferably in the first feeding after spraying. By the joint selection of the Eimeria species and the ionophore such that the former is at least partially resistant to the latter, the severity of the infection is controlled such that feed conversion is optimized yet an immune response still develops. All ionophores exhibit anticoccidial activity, although the relative degree of activity varies among ionophores, and occasionally the toxicity of a particular ionophore may make its use as an anticoccidial impractical. Ionophores of demonstrated commercial use in the control of coccidiosis include lasalocid, salinomycin, monensin, and narasin. Other representative ionophores that may be used in the immunization program include nicarbazin, semduramicin, maduramicin, laidlomycin, nigericin, grisorixin, dianemycin, lenoremycin, lonomycin, X206, alborixin, septamycin, A204, etheromycin, isolasolacid, lysocellin, A23187, A80190 and A80438, beauvericin, nonactin, valinomycin, alixarene, calcimycine, and gramicidin.

Depending on the ionophore chosen, ionophore may be administered at 20-115 g/lb feed, and is preferably administered at the lowest effective dose for each specific ionophore. Ionophore doses and dose ranges currently authorized by the U.S. Food and Drug Administration are shown in Table 1. The ionophore is preferably administered to the fowl in feed.

Coccidial oocysts propagate in infected fowl and are excreted into the fowl's bedding material, a process known as “oocyst shed.” The peak level of coccidial replication generally occurs between 15-24 days of age in a fowl, and the number of shed oocysts is maximal, in general, at about 28 days after infection of the fowl with Eimeria. Because the shed oocysts are infective, it is desirable to prevent high levels of oocyst shed. However, it is also important to maintain the Eimeria infection long enough for an immune response to develop, which requires at least 14-21 days. Therefore, daily administration of ionophore is continued for up to 21 days, and, preferably, for up to anytime between 13 to 18 days. This ionophore treatment prevents the coccidiosis infection from becoming too severe and reduces oocyst shed, but allows the fowl to develop immunity to the Eimeria.

TABLE 1
Authorized dose ranges for ionophores

| | Dose range |
|---|---|
| Ionophore | g/lb feed |
| Lasalocid | 68–113 |
| Salinomycin | 40–60 |
| Monensin | 90–110 |
| Narasin | 54–90 |
| Semduramicin | 22.7 |
| Maduramicin | .002–.004 |

Once the ionophore treatment is discontinued, the fowl are treated for 3 to 10 days with an effective dose of one or more chemotherapeutic anticoccidial agent(s), to control the severity of the coccidial infection and prevent new infections. As a result of limiting the term of this treatment stage, there is less damage to the gastrointestinal tract of the fowl and the Eimeria develops resistance to the chemical agent more slowly than it would otherwise. Preferred anticoccidial agents include diclazuril, nicarbazin, robenidine, halofuginone, zoalene, amprolium, and roxarsone. Other anticoccidial drugs may include arprinocid, clarithromycin, clindamycin, clopidol, decoquinate, letrazuirl, spiramycin, toltrazuril, and sulfa drugs. The anticoccidial agent may be administered in feed or in drinking water. If two or more agents are administered in combination, they may be administered in any combination of feed and/or drinking water, such as a first agent in feed and a second agent in drinking water.

A growth-promoting antibiotic, such as bambermycin, virginiamycin, bacitracin-MD, lincomycin, penicillin, and tetracycline, may be administered in an authorized dose in combination with the anticoccidial agent. However, as shown in Example 3, administration of a growth-promoting antibiotic may have little or no effect on control of coccidiosis in the vaccination program. Therefore, in a preferred embodiment, no growth-promoting antibiotic is given to the fowl.

After the chemotherapeutic agent is withdrawn, ionophore administration is resumed for a period of at least 7 days, and preferably between 7 to 10 days, to protect any fowl that may not yet have developed complete protective immunity. According to a preferred embodiment of the present invention, ionophore administration is not continued throughout the life of the fowl.

### Definitions

Challenge—administering coccidia to fowl to induce coccidiosis.
Feed conversion—amount (in pounds (lbs)) of feed required to produce one pound of meat.
Percent livability=(Number of fowl placed in each pen on day of age)/(Number of fowl per pen moved to processing plant at market age)×100%.
Percent mortality=(Number of fowl that die during experiment)/(Number of fowl placed in pen at start of experiment)×100%.
WAFC—weight adjusted feed conversion. Chickens become less efficient at feed conversion as they mature and become heavier. Standardized conversion factors have accordingly been calculated to adjust feed conversion values to the weight of the fowl. WAFC represents the number of pounds of feed required to produce one pound of meat, adjusted to a standardized weight. Because WAFC increases with the severity of coccidiosis, WAFC is used as a metric to evaluate the level of protection afforded by vaccination or by an anticoccidial drug.

### EXAMPLES

1. Effect of Ionophore Alone at Single Concentration Compared with Ionophore Plus Coccidiostatic Drug on Vaccination Program

All broiler chicks were vaccinated within 24 hours of hatching (day 0) by spraying with a suspension of E. acervulina and E. maxima sporulated oocysts. The is chickens were then reared in three standard broiler houses containing used sawdust as the bedding material to provide an adequate coccidial challenge. Each house was divided into two large pens with 10,800 chickens placed into each pen. One pen within each house was designated as the control group and the other pen was designated as the experimental group. Consequently, there were three replicates of each treatment, providing data on 32,400 birds per treatment group. The control group received the anticoccidial ionophore, monensin, in the feed at 90 grams/ton of feed from day 0 to day 42. The experimental group received monensin, in the feed at 90 grams/ton of feed from day 0 to day 13, the coccidostatic drug, nicarbazin, at 113.5 grams/ton of feed from day 14 to day 17, and then monensin from day 18 to day 42. No anticoccidial compounds were fed to either group from day 43 to day 52.

The results of this experiment are shown in Table 2. The weight-adjusted feed conversion (WAFC) for the experimental group was 0.026 pounds lower than the control group.

TABLE 2

| | # Birds | % | Body | | |
|---|---|---|---|---|---|
| | Placed | Livability | Weight | FC | WAFC* |
| Control | 32,400 | 95.8 | 5.52 | 2.066 | 2.059 |
| Trial | 32,400 | 95.8 | 5.53 | 2.041 | 2.033 |
| p = | | 0.99 | 0.59 | 0.20 | 0.16 |

*WAFC adjusted to 5.45 lb. standard weight using 10:1 ratio

2. Effect of Ionophore Alone at Two Concentrations Compared with Ionophore Plus Coccidiostatic Drug on Vaccination Program

Broiler chicks were vaccinated, divided into groups, and reared as described in the previous example The control group received the anticoccidial ionophore, salinomycin, in feed at 60 grams/ton from day 0 to day 28, followed by salinomycin at 40 grams/ton from day 29 to day 41. The experimental group received salinomycin, in feed at 60 grams/ton from day 0 to day 13, the anticoccidial drug, diclazuril, from day 14 to day 17, salinomycin from day 18 to day 28, and salinomycin at 40 grams/ton from day 29 to day 41. No anticoccidial compounds were fed to either group from day 42 to day 52.

The results are shown in Table 3. The WAFC for the experimental group was 0.023 lower than the control group. The average body weight on day 28 was 0.15 pounds greater for the experimental group, indicating that this group utilized less of its food intake than the control group in fighting the coccidial infection.

TABLE 3

| | | | Body | Body | | |
|---|---|---|---|---|---|---|
| | # Birds | % | Weight | Weight | | |
| | Placed | Livability | 52 days | 28 days | FC | WAFC* |
| Control | 32,400 | 95.4 B | 5.45 A | 2.05 | 2.011 A | 2.015 |
| Trial | 21,600 | 96.2 A | 5.55 A | 2.20 | 1.996 A | 1.992 |
| p = | | 0.04 | 0.17 | | 0.42 | 0.15 |

*WAFC adjusted to 5.50 lb. standard weight using 10:1 ratio

3. Effect of a Growth-Promoting Antibiotic on Vaccination Program

All broiler chicks were vaccinated as described in the two preceding examples. The chickens were then reared in a small pen research house containing used sawdust as the bedding material to provide an adequate coccidial challenge. The trial began with 106 chickens placed into each of 24 pens to provide 6 replicates for each of 4 experimental treatments (total of 636 chickens per treatment group). In Experiment F-68, chickens received an anticoccidial ionophore (narasin), a growth promoting antibiotic (bacitracin-MD (BMD)), and an anticoccidial drug (3-Nitro (roxarsone)) in feed. In Experiment F-69, chickens received only the growth promoting antibiotic, BMD, in feed. In Experiment F-70, the anticoccidial ionophore (narasin) and the anticoccidial drug (3-Nitro (roxarsone)) were given in the feed. In Experiment F-71, no anticoccidial ionophore, growth promoting antibiotic, or anticoccidial drug was administered. Dosages and treatment schedules are shown in Table 4.

TABLE 4

| | | Drug Type and Dose (grams/ton) by Feed Type | | | | |
|---|---|---|---|---|---|---|
| | | Starter | Grower 1 | Grower 2 | Withdrawal I | Withdrawal II |
| Expt. | Cmpd | 0–18 days | 19–27 days | 28–36 days | 37–42 days | 43–50 days |
| F-68 | C-Stat | Narasin 54 | Narasin 54 | Narasin 54 | Narasin 54 | — |
| | GP | BMD 50 | BMD 25 | BMD 25 | BMD 25 | ZBac 25 |
| | 3-Nitro | 3-Nitro 45 | 3-Nitro 45 | 3-Nitro 45 | None | — |
| F-69 | C-Stat | — | — | — | — | — |
| | GP | BMD 50 | BMD 25 | BMD 25 | BMD 25 | ZBac 25 |
| | 3-Nitro | — | — | — | — | — |
| F-70 | C-Stat | Narasin 54 | Narasin 54 | Narasin 54 | Narasin 54 | — |
| | GP | — | — | — | — | — |
| | 3-Nitro | 45 | 45 | 45 | — | — |
| F-71 | C-Stat | — | — | — | — | — |
| | GP | — | — | — | — | — |
| | 3-Nitro | — | — | — | — | — |

Narasin = Monteban;
3-Nitro = roxarsone;
GP = growth promoting antibiotic;
BMD = bacitracin-MD;
ZBac = zinc bacitracin;
C-Stat = coccidiostat

Birds in all treatment groups were subjected to a coccidial challenge at 28 days of age by supplying feed sprayed with an infective dose of coccidial oocysts. Results are shown in Table 5. The coccidiostatic compounds had a significant effect on WAFC, which declined by 0.049 pounds in groups receiving these compounds (F-68 and F-70) compared with groups that were not given coccidiostats (F-69 and F-71). No further improvement in WAFC was achieved by administering a growth promoting antibiotic in addition to the coccidostatic compounds (compare F-68 to F-70).

TABLE 5

| Expt. | Vaccine | C-stat | GPA | Final No. Birds | Mort. % | Avg. Wt. Lbs | FC | WAFC* |
|---|---|---|---|---|---|---|---|---|
| F-68 | Yes | Yes | Yes | 594 | 3.6 | 5.31 | 1.922 | 1.921 |
| F-69 | Yes | No | Yes | 589 | 4.6 | 5.26 | 1.948 | 1.952 |
| F-70 | Yes | Yes | No | 590 | 4.6 | 5.32 | 1.898 | 1.897 |
| F-71 | Yes | No | No | 587 | 4.4 | 5.25 | 1.960 | 1.965 |
| | | | | p = | | 0.40 | <0.05** | 0.001** |

COCCIDIOSTAT:

| | | | | | |
|---|---|---|---|---|---|
| Yes (F-68/70) | 1184 | 4.1 | 5.31 | 1.910 | 1.909 |
| No (F-69/71) | 1176 | 4.5 | 5.25 | 1.954 | 1.958 |
| | p = | | 0.08 | <0.05 | <0.05 |

GROWTH PROMOTANT: (GPA)

| | | | | | |
|---|---|---|---|---|---|
| Yes (F-68/69) | 1183 | 4.1 | 5.28 | 1.935 | 1.937 |
| No (F-70/71) | 1177 | 4.5 | 5.28 | 1.929 | 1.931 |
| | p = | | 0.97 | 0.655 | 0.695 |

*WAFC adjusted to 5.30 lb. standard weight using 10:1 ratio
**significant for groups 69 vs. 70, 70 vs. 71, and 68 vs. 71

While preferred embodiments of the invention have been shown and described herein, it will be understood that such embodiments are provided by way of example only. Numerous variations, changes and substitutions will occur to those having ordinary skill in the art without departing from the spirit of the invention. Accordingly, it is intended that the appended claims cover all such variations that fall within the spirit and scope of the invention.

1. A method for protecting a fowl against coccidiosis comprising the steps of:
(a) orally inoculating a neonatal fowl with a suspension of sporulated coccidial oocysts, wherein the coccidial oocysts are from a strain of Eimeria that is at least partially resistant to an ionophore;
(b) subsequently administering to the inoculated fowl the ionophore to which the Eirneria strain is at least partially resistant;
(c) continuing to administer the ionophore to the inoculated fowl daily for up to 21 days;
(d) subsequently administering at least one chemotherapeutic anticoccidial agent to the inoculated fowl for 3 to 10 days; and
(e) subsequently administering the ionophore to the inoculated fowl daily for at least 7 days.

2. The method of claim 1, wherein the fowl are inoculated by spraying with a suspension of sporulated coccidial oocysts within 48 hours of hatching.

3. The method of claim 2, wherein the spraying occurs within 12 to 24 hours of hatching.

4. The method of claim 1, wherein the coccidial oocysts are obtained from Eirneria species selected from the group consisting of Eirneria maxima, Eirneria acervulina, Eirneria tenella, Eirneria mivati, and Eirneria necatrix and combinations thereof.

5. The method of claim 1, wherein the suspension of coccidial oocysts comprises 10 to 10,000 sporulated oocysts per dose.

6. The method of claim 5, wherein the suspension of coccidial oocysts comprises 100 to 1,000 sporulated oocysts per dose.

7. The method of claim 1, wherein the ionophore is selected from the group consisting of lasalocid, salinomycin, monensin, narasin, nicarbazin, semduramicin, and maduramicin.

8. The method of claim 1, wherein the ionophore is administered in feed in an amount ranging from 20 to 115 grams per ton of feed.

9. The method of claim 1, wherein the at least one anticoccidial agent comprises an effective dose of at least one drug selected from the group consisting of diclazuril, nicarbazin, robenidine, hatofuginone, zoalene, amprolium, and roxarsone.

10. The method of claim 1, wherein the fowl is selected from the group consisting of chickens, turkeys, ducks, geese, quail, and pheasants.

11. The method of claim 1, wherein the ionophore is administered in feed.

12. The method of claim 1, wherein the at least one anticoccidial agent is administered in feed or in drinking water.

13. The method of claim 12, wherein the at least one anticoccidial agent comprises a first anticoccidial agent and a second anticoccidial agent, and wherein the first anticoccidial agent is administered in the feed and the second anticoccidial agent is administered in the drinking water.

14. The method of claim 7, wherein the ionophore is narasin or a combination of narasin and nicarbazin in equal amounts.

15. The method of claim 14, wherein the ionophore is administered at about 54 to 90 grams per ton of feed.

16. The method of claim 7, wherein the ionophore is lasalocid.

17. The method of claim 16, wherein the ionophore is administered at about 20 to 115 grams per ton of feed.

18. The method of claim 7, wherein the ionophore is salinomycin.

19. The method of claim 18, wherein the ionophore is administered at about 40 to 60 grams per ton of feed.

20. The method of claim 7, wherein the ionophore is monensin.

21. The method of claim 20, wherein the ionophore is administered at about 90 to 110 grams per ton of feed.

22. The method of claim 7, wherein the ionophore is semduramicin.

23. The method of claim 22, wherein the ionophore is administered at about 20 to 23 grams per ton of feed.

24. A method for protecting a fowl against coccidiosis consisting of the steps of:
(a) orally inoculating a neonatal fowl with a suspension of sporulated coccidial oocysts, wherein the coccidial oocysts are from a strain of Eimeria that is at least partially resistant to an ionophore;
(b) subsequently administering to the inoculate fowl the ionophore to which the Eimeria strain is at least partially resistant;
(c) continuing to administer the ionophore to the inoculated fowl daily for up to 21 days;
(d) subsequently administering at least one chemotherapeutic anticoccidial agent to the inoculated fowl for 3 to 10 days; and
(e) subsequently administering the ionophore to the inoculated fowl daily for at least 7 days.
